(19) United States
(12) Patent Application Publication
(10) Pub. No.: US 2022/0323139 A1
(43) Pub. Date: Oct. 13, 2022

(54) MEDICAL DEVICE, METHOD FOR CONTROLLING MEDICAL DEVICE, AND CONTROL DEVICE

(71) Applicant: OLYMPUS CORPORATION, Tokyo (JP)

(72) Inventor: Hiroyuki TAKAYAMA, Tokyo (JP)

(73) Assignee: OLYMPUS CORPORATION, Tokyo (JP)

(21) Appl. No.: 17/843,266

(22) Filed: Jun. 17, 2022

Related U.S. Application Data
(63) Continuation of application No. PCT/JP2020/009898, filed on Mar. 9, 2020.

(51) Int. Cl. A61B 18/14

(52) U.S. Cl. CPC A61B 18/1445; A61B 2017/2943

(57) ABSTRACT

A medical device includes a pair of jaws that grips biological tissue, is coupled at first ends of the jaws, opens and closes as a result of swiveling of at least one of the jaws, and comprises a pair of gripping surfaces facing when the jaws are closed, a motor that generates a closing driving force for closing the jaws, a driving-force transmission member that connects the jaws to the motor and that is configured to transmit the closing driving force from the motor to at least one of the jaws, and a controller that is connected to the motor and that controls the motor. The controller temporally changes magnitude of the closing driving force generated by the motor so as to shift peak positions of gripping pressure for gripping the biological tissue in the pair of gripping surfaces.

### CROSS-REFERENCE TO RELATED APPLICATIONS

This is a continuation of International Application PCT/JP2020/009898 which is hereby incorporated by reference herein in its entirety.

### TECHNICAL FIELD

The present embodiment relates to medical devices, methods for controlling medical devices, and control devices.

### BACKGROUND ART

A known medical device in the related art is used for treating biological tissue gripped by a pair of jaws (e.g., see Patent Literatures 1 and 2).

### CITATION LIST

### Patent Literature

### {PTL 1}

Japanese Unexamined Patent Application, Publication No. 2010-51802

### {PTL 2}

Japanese Unexamined Patent Application, Publication No. 2019-136510

### SUMMARY OF INVENTION

A first aspect of the present invention provides a medical device including: a pair of jaws that grips biological tissue, is coupled at first ends of the pair of jaws, opens and closes as a result of swiveling of at least one of the jaws, and comprises a pair of gripping surfaces facing when the pair of jaws is closed; a motor that generates a closing driving force for closing the pair of jaws; a driving-force transmission member that connects the pair of jaws to the motor and that is configured to transmit the closing driving force from the motor to at least one of the pair of jaws; and

a controller that is connected to the motor and that controls the motor. The controller temporally changes magnitude of the closing driving force generated by the motor so as to shift peak positions of gripping pressure for gripping the biological tissue in the pair of gripping surfaces.

A second aspect of the present invention provides a method for controlling a medical device including a pair of jaws that grips biological tissue. The pair of jaws is coupled at first ends of the pair of jaws, opens and closes as a result of receiving a driving force from a motor and comprises a pair of gripping surfaces facing when the pair of jaws is closed. The method includes temporally changing magnitude of a closing driving force for closing the pair of jaws so as to shift peak positions of gripping pressure for gripping the biological tissue in the pair of gripping surfaces.

A third aspect of the present invention provides a control device controlling a medical system. The medical system includes a pair of jaws that grips biological tissue, is coupled at first ends of the pair of jaws, opens and closes as a result of swiveling of at least one of the jaws, and comprises a pair of gripping surfaces facing when the pair of jaws is closed, a motor that generates a closing driving force for closing the pair of jaws and a driving-force transmission member that connects the pair of jaws to the motor and that is configured to transmit the closing driving force from the motor to at least one of the pair of jaws. The control device includes a processor including hardware. The processor is configured to temporally change magnitude of the closing driving force generated by the motor so as to shift peak positions of gripping pressure for gripping the biological tissue in the pair of gripping surfaces.

### BRIEF DESCRIPTION OF DRAWINGS

FIG. 1 is an external view of a medical device according to an embodiment of the present invention.

FIG. 2 schematically illustrates the configuration of a device body of a medical device according to a first embodiment of the present invention.

FIG. 3 is a block diagram of the medical device according to the first embodiment of the present invention.

FIG. 4A is a side view schematically illustrating a pair of jaws that is closed to a position in which gripping surfaces are parallel to each other.

FIG. 4B illustrates the pair of jaws and a spatial distribution of gripping pressure when a low gripping force is applied thereto.

FIG. 4C illustrates the pair of jaws and a spatial distribution of gripping pressure when a high gripping force is applied thereto.

FIG. 5A is a table showing an example of the relationship between a gripping force and a time proportion.

FIG. 5B is a graph showing the relationship between a gripping force and gripping pressure.

FIG. 5C is a graph showing an example of a temporal change in the gripping force.

FIG. 5D illustrates an example of a gripping-force target curve.

FIG. 6A is a flowchart illustrating a method for controlling the medical device in FIGS. 2 and 3.

FIG. 6B is a flowchart illustrating an incision routine in the flowchart in FIG. 6A.

FIG. 7 schematically illustrates the configuration of a device body of a medical device according to a second embodiment of the present invention.

FIG. 8 is a block diagram of the medical device according to the second embodiment of the present invention.

FIG. 9 illustrates shifting of a driving-force transmission member as an incision process performed on biological tissue progresses.

FIG. 10 is a flowchart illustrating an incision routine in the method for controlling the medical device in FIGS. 7 and 8.

FIG. 11 illustrates a method for setting a threshold value for a positional deviation amount of the proximal end of the driving-force transmission member.

FIG. 12 is a block diagram of a medical device according to a third embodiment of the present invention.

FIG. 13 is a flowchart illustrating an incision routine in the method for controlling the medical device in FIG. 12.

FIG. 14A illustrates a spatial distribution of gripping pressure when thick tissue is being gripped by the pair of gripping surfaces.

FIG. 14B illustrates a spatial distribution of gripping pressure when tissue is being gripped by a part of the pair of gripping surfaces.

### DESCRIPTION OF EMBODIMENTS

### First Embodiment

A medical device according to a first embodiment of the present invention will now be described with reference to the drawings.

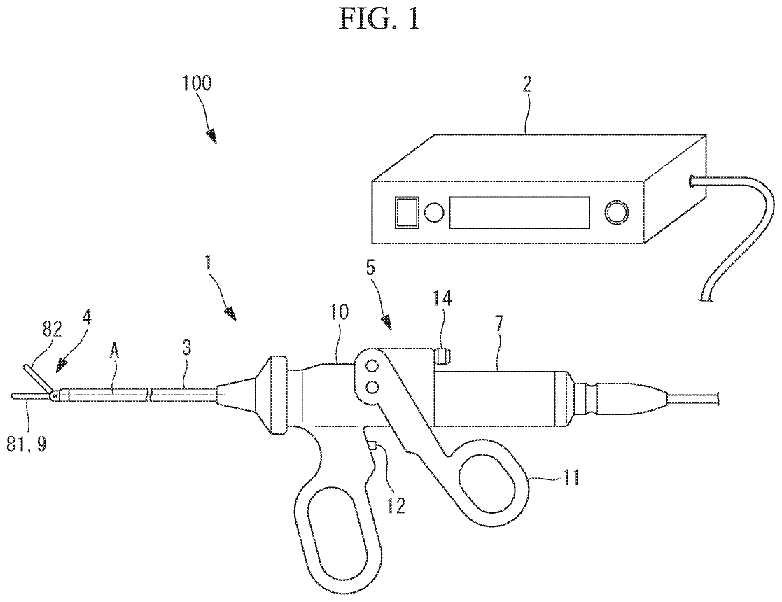

As shown in FIG. 1, a medical device 100 according to this embodiment includes a device body 1 that grips and treats biological tissue S and a controller 2 that is connected to the device body 1 and that controls the device body 1.

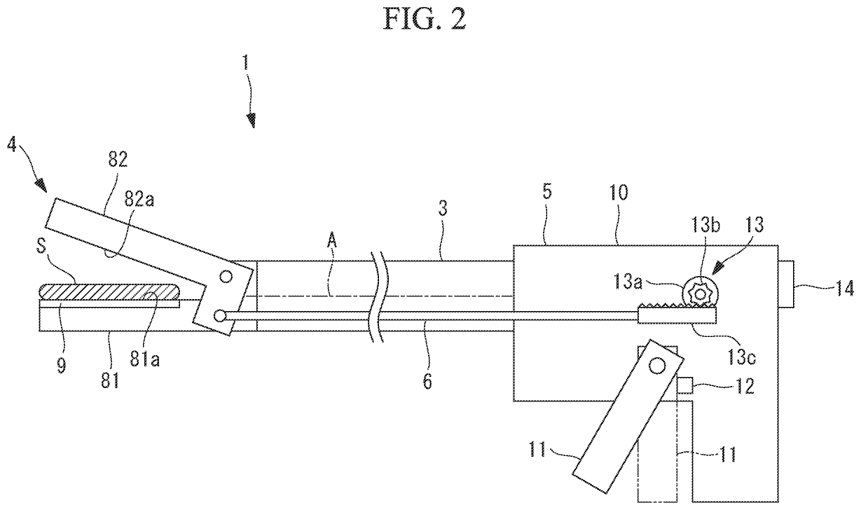

The device body 1 includes a long shaft 3, an end effector 4 connected to the distal end of the shaft 3, a driving operation unit 5 connected to the proximal end of the shaft 3, a driving-force transmission member 6 (see FIG. 2) extending through the shaft 3 from the driving operation unit 5 to the end effector 4, and an energy unit 7 connected to the driving operation unit 5.

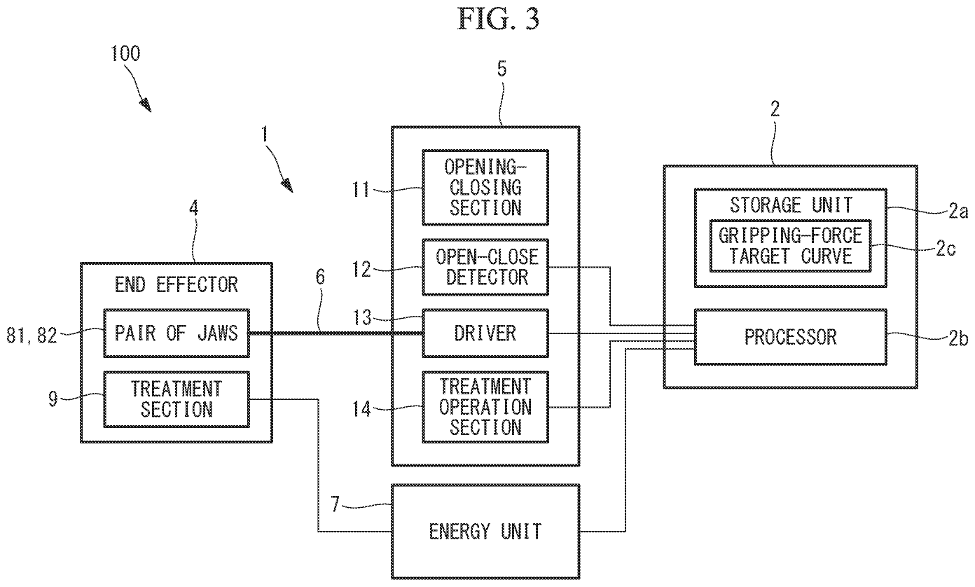

As shown in FIGS. 2 and 3, the end effector 4 includes a pair of jaws 81 and 82 and a treatment section 9 provided on at least one of the pair of jaws 81 and 82. The pair of jaws is including a jaw 81 (a first jaw 81) and a jaw 82 (a second jaw 82).

The proximal ends of a first jaw 81 and a second jaw 82 are coupled to each other in a swivelable manner about a swivel axis extending orthogonally to a longitudinal axis A of the shaft 3. The swiveling of the pair of jaws 81 and 82 cause the distal ends of the pair of jaws 81 and 82 to open and close. The pair of jaws 81 and 82 respectively comprises gripping surfaces 81a and 82a that face each other when the pair of jaws 81 and 82 is closed, and grips the tissue S between the gripping surface 81a (the first gripping surface 81a) and the gripping surface 82a (the second gripping surface 82a).

In this embodiment, the pair of jaws 81 and 82 is of a single-swiveling type in which the jaw 81 is a stationary jaw fixed to the shaft 3 and the jaw 82 is a movable jaw that is swivelable. The pair of jaws 81 and 82 may alternatively be of a double-swiveling type in which the jaws 81 and 82 are both swivelable.

Each of the jaws 81 and 82 has a small diameter and has rigidity low enough to be bendable by a gripping force F applied to the movable jaw 82, as will be described later.

The treatment section 9 applies energy for incising the tissue S to the tissue S between the gripping surfaces 81a and 82a. For example, the treatment section 9 is an ultrasonic probe that outputs ultrasonic energy, a heater that output thermal energy, or a high-frequency electrode that outputs high-frequency energy. The treatment section 9 is connected to the energy unit 7 via the shaft 3 and the driving operation unit 5 and generates energy by being supplied with an energy source, such as high-frequency power, from the energy unit 7. Thus, the energy unit 7 has an energy-source generating device, such as a power source device.

Instead of incising the biological tissue S by using energy, the treatment section 9 may mechanically incise the biological tissue S by using a blade. Specifically, the end effector 4 may be scissors forceps obtained by providing the pair of jaws 81 and 82 with blades as the treatment section 9.

Furthermore, the treatment section 9 may perform treatment other than an incision process on the biological tissue S. For example, the treatment section 9 may apply energy to the biological tissue S to seal the biological tissue S.

The driving operation unit 5 includes a main body 10 fixed to the proximal end of the shaft 3, and also includes an opening-closing section 11, an open-close detector 12, a driver 13, and a treatment operation section 14 that are provided at the main body 10.

The opening-closing section 11 is to be operated by an operator for opening and closing the pair of jaws 81 and 82 and is shiftable between an open position and a closed position relative to the main body 10. In this embodiment, the opening-closing section 11 is a handle that swivels from the open position to the closed position relative to the main body 10 by being gripped by the operator. Alternatively, the opening-closing section 11 may be a push button or a toggle switch.

As an alternative to FIG. 1 in which the handle 11 is gripped toward the distal end, the handle 11 may be gripped toward the proximal end, as shown in FIGS. 2 and 3.

The open-close detector 12 detects whether or not the handle 11 is in the closed position, transmits a closed signal to the controller 2 when the handle 11 is in the closed position, and transmits an open signal to the controller 2 when the handle 11 is in a position other than the closed position. For example, the open-close detector 12 is a detection switch that is turned on and off by the handle 11. When the handle 11 is in the closed position, the detection switch is turned on to output a closed signal. When the handle 11 is in a position other than the closed position, the detection switch is turned off to output an open signal.

The driver 13 is an actuator that is connected to the proximal end of the driving-force transmission member 6 and that shifts the driving-force transmission member 6 in the longitudinal direction. In detail, the driver 13 includes a motor (i.e., a driving source) 13a that generates torque as a driving force for opening and closing the pair of jaws 81 and 82, and also includes a pinion 13b and a rack 13c that convert the torque generated by the motor 13a into a rectilinear motion in the direction parallel to the longitudinal axis A of the driving-force transmission member 6.

The driving-force transmission member 6 is a long member disposed inside the shaft 3 and extending in a direction parallel to the longitudinal axis A of the shaft 3, and is, for example, a rod, a highly-rigid rod, a wire or a flexible wire. The distal end of the driving-force transmission member 6 is fixed to the proximal end of the movable jaw 82, and the proximal end of the driving-force transmission member 6 is fixed to the rack 13c.

The driver 13 generates torque as a closing driving force f for closing the pair of jaws 81 and 82. The proximal end of the driving-force transmission member 6 is pulled by the closing driving force f, and the gripping force F that causes the movable jaw 82 to swivel in the closing direction is transmitted from the driving-force transmission member 6 to the movable jaw 82. With regard to the magnitude of the gripping force F, the gripping force F increases as the closing driving force f increases based on the magnitude of the closing driving force f and the power transmission efficiency of the driving-force transmission member 6.

Furthermore, as an opening driving force for opening the pair of jaws 81 and 82, the driver 13 generates torque in the opposite direction from the closing driving force. The proximal end of the driving-force transmission member 6 is pressed by the opening driving force, and a releasing force that causes the movable jaw 82 to swivel in the opening direction is transmitted from the driving-force transmission member 6 to the movable jaw 82.

The treatment operation section 14 is to be operated by the operator for commencing and terminating an incision process on the tissue S. In one example, the treatment operation section 14 is an input device, such as a push button switch, a foot pedal, or an audio input device. When the operator performs an operation for commencing an incision process, the treatment operation section 14 transmits a treatment-section driving signal to the controller 2 in response to, for example, pressing of the switch. The controller 2 controls the energy unit 7 in response to the treatment-section driving signal, so that an energy source is supplied from the energy unit 7 to the treatment section 9, thereby incising the tissue S.

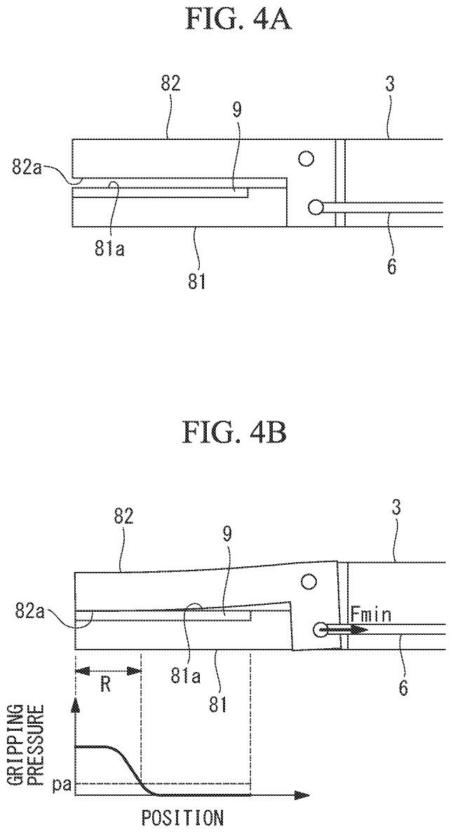

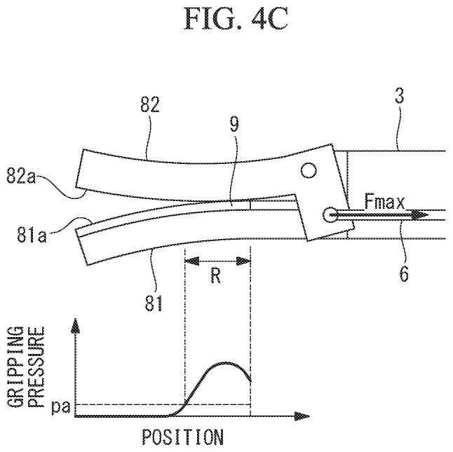

In order to incise the tissue S by efficiently applying energy from the treatment section 9 to the tissue S between the gripping surfaces 81a and 82a, the gripping surfaces 81a and 82a can grip the tissue S with a required gripping pressure larger than a target gripping pressure pa. As shown in FIGS. 4B and 4C, a region R where the required gripping pressure is generated shifts in accordance with the magnitude of the gripping force F applied to the movable jaw 82, thus causing a region where the tissue S is incised to shift accordingly.

In detail, as shown in FIG. 4A, in a state where the gripping surfaces 81a and 82a are parallel to each other, a gap is formed between the gripping surfaces 81a and 82a. Therefore, as shown in FIG. 4B, when a low gripping force Fmin is applied to the movable jaw 82, the distal ends of the gripping surfaces 81a and 82a come into contact with each other, such that the required gripping pressure is generated between the distal ends of the gripping surfaces 81a and 82a. In this case, the tissue S between the distal ends of the gripping surfaces 81a and 82a is incised. On the other hand, as shown in FIG. 4C, when a high gripping force Fmax is applied to the movable jaw 82, the proximal ends of the gripping surfaces 81a and 82a come into contact with each other, such that the required gripping pressure is generated between the proximal ends of the gripping surfaces 81a and 82a. In this case, the tissue S between the proximal ends of the gripping surfaces 81a and 82a is incised.

Accordingly, the bending of the jaws 81 and 82 increases with increasing gripping force F, so that the region R where the required gripping pressure is generated shifts from the distal ends toward the proximal ends of the gripping surfaces 81a and 82a.

In a case where each jaws intersect each other like a pair of scissors, the relationship between the magnitude of the closing driving force and the region R is inverted. In other words, as the closing driving force and the gripping force increase, the region R shifts from the proximal ends toward the distal ends of the gripping surfaces 81a and 82a.

As shown in FIG. 3, the controller 2 includes a storage unit 2a having a RAM (random access memory), a ROM (read-only memory), and another freely-chosen memory, and also includes a processor (control unit) 2b, such as a central processing unit.

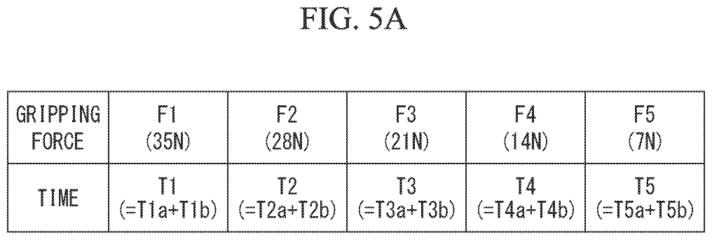

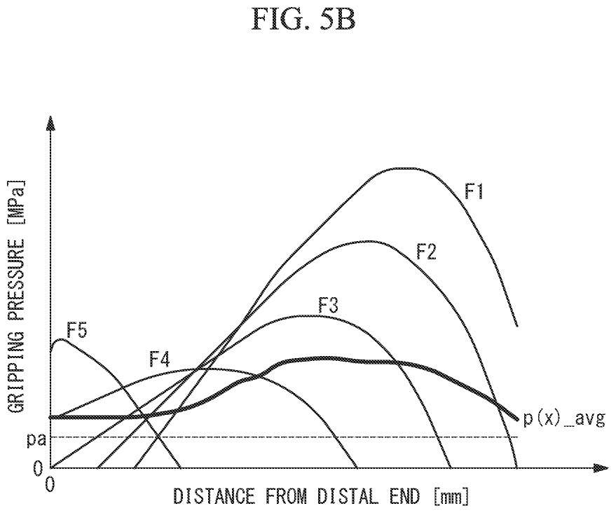

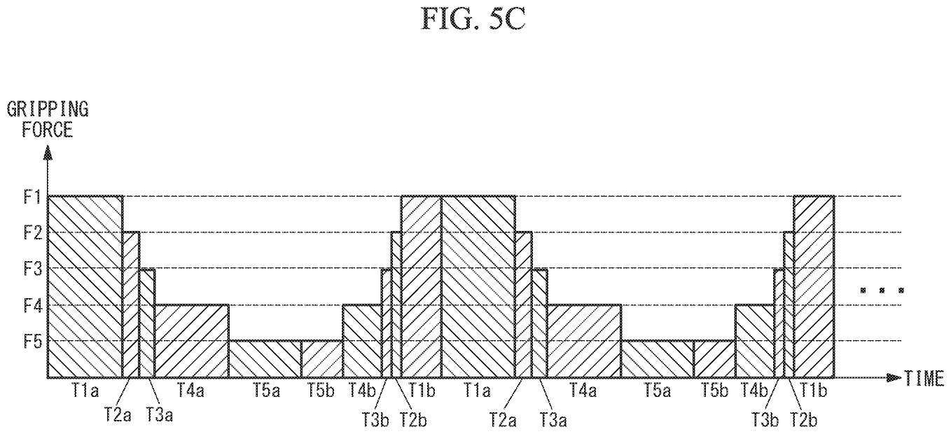

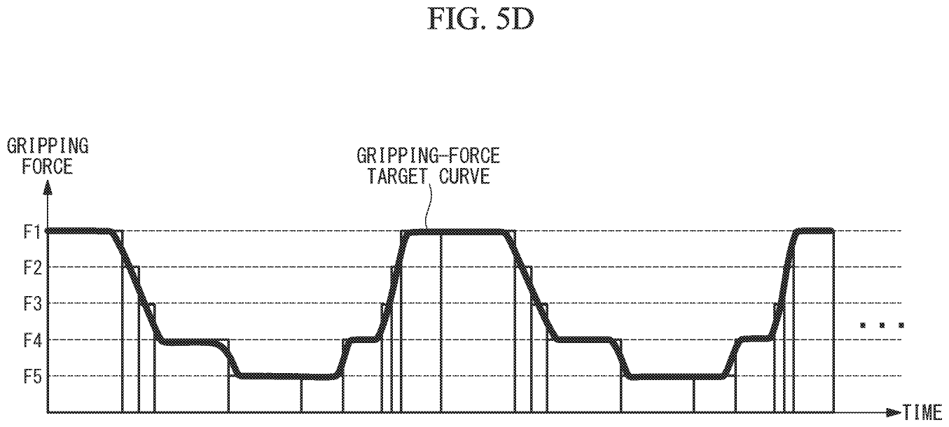

As shown in FIG. 5D, the storage unit 2a has stored therein a gripping-force target curve 2c that indicates a temporal change pattern of the magnitude of the gripping force F. In this temporal change pattern, the magnitude of the gripping force F temporally fluctuates between the low gripping force Fmin (F5) and the high gripping force Fmax (F1).

The processor 2b controls the driver 13 and the energy unit 7 based on the open signal and the closed signal from the open-close detector 12 and the treatment-section driving signal from the treatment operation section 14, so as to cause the pair of jaws 81 and 82 to grip and release the tissue S and to cause the treatment section 9 to incise the tissue S. Furthermore, the processor 2b temporally changes the magnitude of the closing driving force f in accordance with the gripping-force target curve 2c during a period in which the tissue S is being incised by the treatment section 9, so as to cause the region R where the required gripping pressure is generated to reciprocate between the distal ends and the proximal ends of the gripping surfaces 81a and 82a.

FIGS. 5A to 5D illustrate an example of a method for setting the gripping-force target curve 2c. This setting method includes a first step for setting time proportions of a plurality of gripping forces F1, F2, F3, F4, and F5 and a second step for setting a gripping-force target curve.

As shown in FIG. 5A, in the first step, time proportions T1, T2, T3, T4, and T5 of the gripping forces F1, F2, F3, F4, and F5 are set for performing a desired incision process on the tissue S. For example, if the tissue S is to be incised over the entire length of the gripping surfaces 81a and 82a, the time proportions T1, T2, T3, T4, and T5 are set such that the gripping pressure is larger than the target gripping pressure pa over the entire length of the gripping surfaces 81a and 82a. The time proportions T1, T2, T3, T4, and T5 are set based on the relationship between the gripping forces F1, F2, F3, F4, and F5 (F1>F2>F3>F4>F5) shown in FIG. 5B and the position of the region R. In FIG. 5B, the abscissa axis indicates the position (i.e., the distance from the distal ends of the gripping surfaces 81a and 82a) in the direction parallel to the longitudinal axis A on the gripping surfaces 81a and 82a, whereas the ordinate axis indicates the magnitude of the gripping pressure at each position of the gripping surfaces 81a and 82a.

In one example, if the gripping forces F1, F2, F3, F4, and F5 are to be temporally changed, as shown in FIG. 5C, an average value p(x)_avg of gripping pressure pi(x) is expressed with Expression (1) indicated below, where pi(x) denotes gripping pressure at a position where the distance from the distal ends of the gripping surfaces 81a and 82a is x. As shown in FIG. 5B, the time proportions T1, T2, T3, T4, and T5 are set such that the average value p(x)_avg is larger than the target gripping pressure pa at all the positions x.

$${p ( x )}_{- avg} = \frac{{\sum }_{i = 1}^{5} pi ( x ) \cdot Ti}{{\sum }_{i = 1}^{5} Ti} \qquad ( 1 )$$

In the second step, a continuous gripping-force target curve 2c that interpolates between the discrete gripping forces F1, F2, F3, F4, and F5 is set, as shown in FIG. 5D. The gripping-force target curve 2c is set in view of the operating performance, such as acceleration and deceleration of the motor 13a of the driver 13.

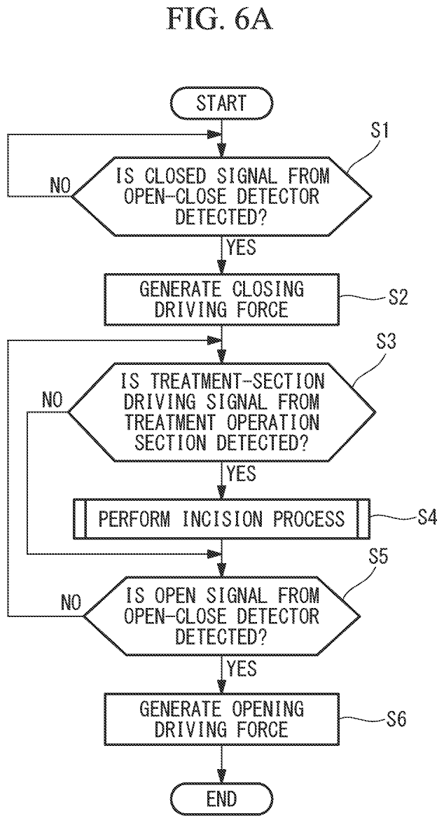

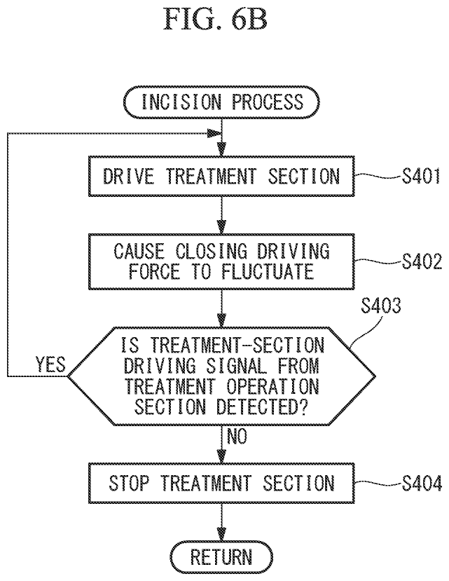

Next, a method of how the medical device 100 is controlled by the processor 2b will be described with reference to FIGS. 6A and 6B. This control method is implemented by the processor 2b executing a process in accordance with a control program (not shown) stored in the storage unit 2a.

As shown in FIG. 6A, when an operator grips the handle 11 from the open position to the closed position, the open-close detector 12 detects that the handle 11 has been gripped, and a closed signal is transmitted from the open-close detector 12 to the processor 2b (YES in step S1). The processor 2b controls the driver 13 in response to the closed signal and causes the driver 13 to generate a closing driving force f (step S2). Accordingly, the closing driving force f is transmitted as a gripping force F to the movable jaw 82 via the driving-force transmission member 6, so that the pair of jaws 81 and 82 closes in accordance with the gripping force F. The magnitude of the closing driving force f in this case is such that, for example, required gripping pressure is generated at the distal ends of the pair of gripping surfaces 81a and 82a.

Subsequently, when the operator operates the treatment operation section 14 to commence an incision process, a treatment-section driving signal is transmitted from the treatment operation section 14 to the processor 2b (YES in step S3). The processor 2b controls the energy unit 7 and the driver 13 in response to the treatment-section driving signal, so as to cause the treatment section 9 to incise the tissue S (step S4).

In detail, as shown in FIG. 6B, the processor 2b causes the energy unit 7 to supply an energy source to the treatment section 9, thereby driving the treatment section 9 (step S401). Accordingly, energy is supplied from the treatment section 9 to the tissue S between the pair of gripping surfaces 81a and 82a.

While controlling the energy unit 7, the processor 2b concurrently controls the driver 13 to cause the magnitude of the closing driving force f to temporally fluctuate between a low driving force (i.e., a second driving force) and a high driving force (i.e., a first driving force) in accordance with the gripping-force target curve 2c (step S402). In other words, the processor 2b converts the magnitude of the gripping force F on the gripping-force target curve 2c to the magnitude of the closing driving force f, and causes the motor 13a to generate the obtained closing driving force f. For the conversion from the gripping force F to the closing driving force f, for example, a preset function with respect to the magnitude of the gripping force F and the magnitude of the closing driving force f is used. Accordingly, the region R where the required gripping pressure is generated reciprocates between the distal ends and the proximal ends of the pair of gripping surfaces 81a and 82a, so that a region where the tissue S is incised by the energy also reciprocates between the distal ends and the proximal ends of the pair of gripping surfaces 81a and 82a.

Until the operator operates the treatment operation section 14 to terminate the incision process (step S403), the supply of energy from the treatment section 9 to the tissue S and the reciprocation of the region R are continuously performed.

The timing at which the incision process is to be terminated may be set arbitrarily by the operator. For example, a treatment detector that detects the degree of opening between the pair of jaws 81 and 82 may be provided. The operator may calculate the thickness of the tissue S between the pair of gripping surfaces 81a and 82a from a detection result obtained by the treatment detector, estimate the progress of the incision process from the thickness, and set the timing for terminating the incision process based on the estimation result.

When the operator operates the treatment operation section 14 to terminate the incision process, the treatment operation section 14 stops transmitting the treatment-section driving signal to the processor 2b (NO in step S403). The processor 2b causes the energy unit 7 to stop supplying the energy source to the treatment section 9 and causes the driver 13 to stop temporally changing the closing driving force f (step S404).

Subsequently, as shown in FIG. 6A, when the handle 11 is released by the operator and moves to the open position, the open-close detector 12 detects that the handle 11 has been released, and an open signal is transmitted from the open-close detector 12 to the processor 2b (YES in step S5). The processor 2b controls the driver 13 in response to the open signal and causes the driver 13 to generate an opening driving force (step S6). Accordingly, the opening driving force is transmitted as a releasing force to the movable jaw 82 via the driving-force transmission member 6, so that the pair of jaws 81 and 82 opens in accordance with the releasing force.

Accordingly, in this embodiment, during a period in which the pair of jaws 81 and 82 is closed and the tissue S is being supplied with energy from the treatment section 9, the temporal fluctuations in the magnitude of the closing driving force f cause the region R where the required gripping pressure is generated to reciprocate continuously between the distal ends and the proximal ends of the pair of gripping surfaces 81a and 82a. Consequently, the required gripping pressure for incising the tissue S is generated over the entire length of the pair of gripping surfaces 81a and 82a, so that the tissue S between the pair of gripping surfaces 81a and 82a can be incised over the entire length.

Furthermore, the pair of jaws 81 and 82 does not necessarily need to be provided with a mechanism for shifting the region R, so that the pair of jaws 81 and 82 used can have a simple structure. Therefore, the jaws 81 and 82 can be readily reduced in diameter.

Moreover, by performing simple control involving causing the closing driving force f to temporally change repeatedly in accordance with a predetermined temporal change pattern, an incision process can be performed over the entire length of the tissue S.

### Second Embodiment

Next, a medical device and a control method therefor according to a second embodiment of the present invention will be described.

In this embodiment, components different from those in the first embodiment will be described, whereas components identical to those in the first embodiment will be given the same reference signs, and descriptions thereof will be omitted.

A medical device 200 according to this embodiment applies the high gripping force Fmax to the movable jaw 82 at the start of an incision process, detects the progress of the incision process performed on the tissue S, and gradually decreases the gripping force F in accordance with the progress of the incision process performed on the tissue S.

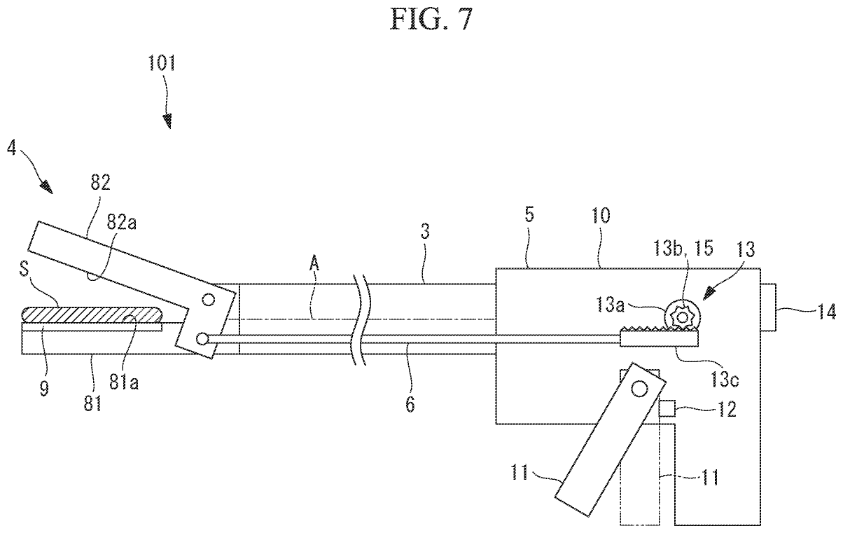

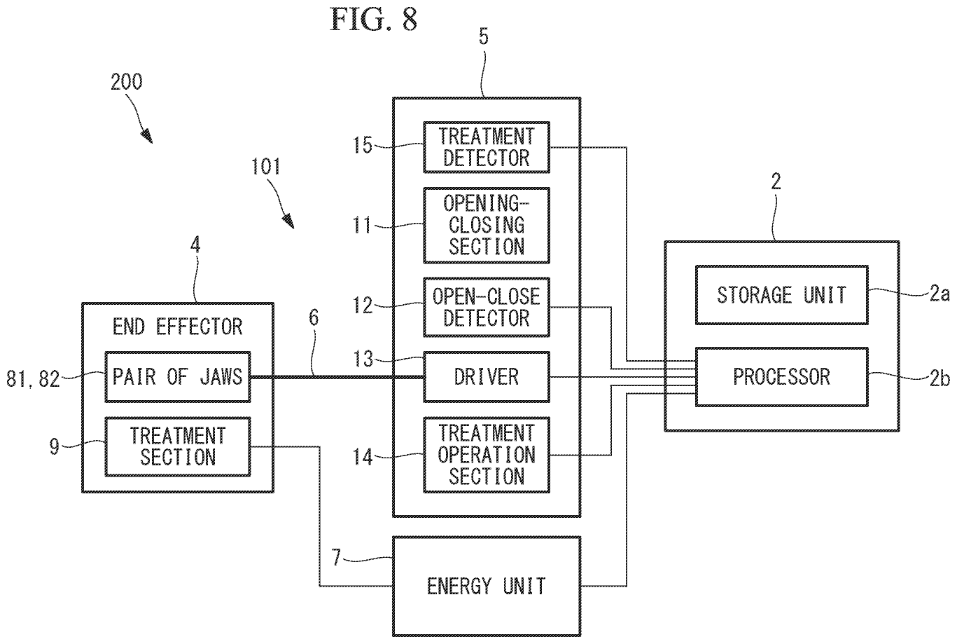

In detail, as shown in FIGS. 7 and 8, the medical device 200 includes a device body 101 and the controller 2. In addition to the shaft 3, the end effector 4, the driving operation unit 5, the driving-force transmission member 6, and the energy unit 7, the device body 101 further includes a treatment detector 15 that detects the progress of the incision process performed on the tissue S by the treatment section 9.

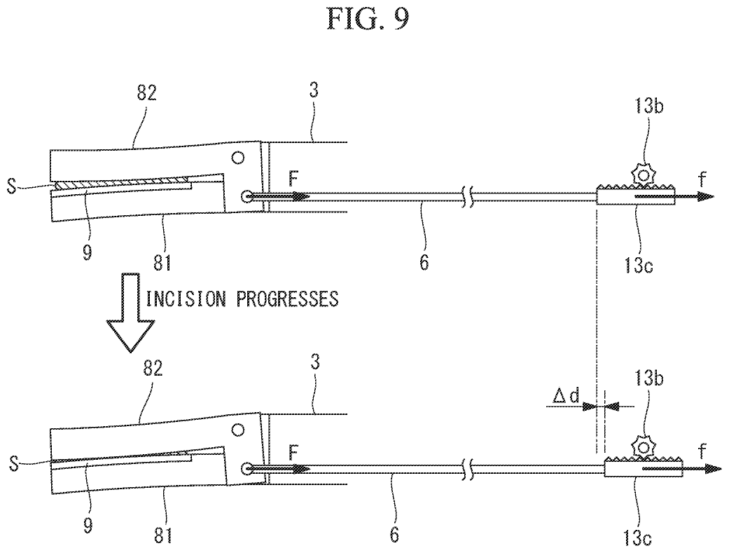

In order to detect the progress of the incision process performed on the tissue S, the treatment detector 15 detects the degree of opening between the pair of jaws 81 and 82. As shown in FIG. 9, in a state where the magnitude of the closing driving force f and the magnitude of the gripping force F are constant, the tissue S decreases in thickness and the pair of jaws 81 and 82 closes further (i.e., the rotational angle of the movable jaw 82 in the closing direction increases) as the incision process performed on the tissue S progresses. Thus, the degree of opening between the pair of jaws 81 and 82 indicates the progress of the incision process performed on the tissue S.

In this embodiment, the treatment detector 15 includes an encoder that detects a position, in the direction parallel to the longitudinal axis A, of the proximal end of the driving-force transmission member 6 as the degree of opening between the pair of jaws 81 and 82. As the tissue S decreases in thickness and the rotational angle of the movable jaw 82 in the closing direction increases, the position of the proximal end of the driving-force transmission member 6 shifts in the direction parallel to the longitudinal axis A. Thus, the progress of the incision process performed on the tissue S can be detected from a deviation amount Δd by which the proximal end of the driving-force transmission member 6 has positionally deviated from the start of the incision process when the closing driving force f and the gripping force F are constant. Together with the proximal end of the driving-force transmission member 6, the position of the rack 13c and the rotational angle of the pinion 13b and the motor 13a also change. For example, the encoder is provided at the motor 13a and indirectly detects the position of the proximal end of the driving-force transmission member 6 from the rotational angle of the motor 13a.

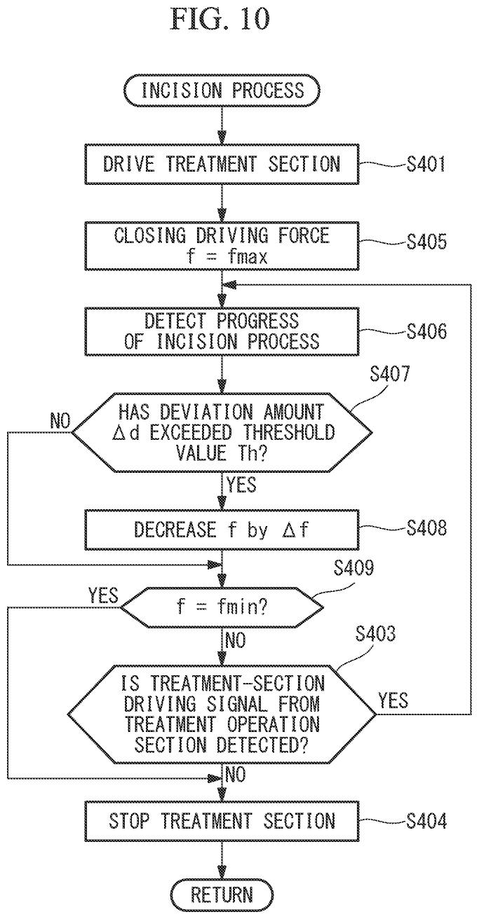

In this embodiment, as shown in FIG. 10, the processor 2b controls the energy unit 7 in response to a treatment-section driving signal and drives the treatment section 9 by causing the energy unit 7 to supply an energy source to the treatment section 9 (step S401).

Furthermore, the processor 2b controls the closing driving force f generated by the motor 13a to a high driving force fmax (step S405). The high driving force fmax corresponds to the high gripping force Fmax at which required gripping pressure is generated at the proximal ends of the pair of gripping surfaces 81a and 82a. Therefore, the tissue S between the proximal ends of the pair of gripping surfaces 81a and 82a is incised first.

The processor 2b monitors the position of the proximal end of the driving-force transmission member 6 detected by the treatment detector 15 during the period in which the treatment section 9 is being driven and the tissue S is being incised (step S406). When the deviation amount Δd at the closing driving force fmax exceeds a predetermined threshold value Th (YES in step S407), the processor 2b decreases the closing driving force f by Δf (step S408). Accordingly, the gripping force F decreases by ΔF, and the region R where the required gripping pressure is generated shifts toward the distal end.

Subsequently, the processor 2b monitors the position of the proximal end of the driving-force transmission member 6 detected by the treatment detector 15 (step S406). In a case where the deviation amount Δd from the start of the incision process when the closing driving force f is equal to fmax−Δf exceeds the threshold value Th (YES in step S407), the processor 2b decreases the closing driving force f by Δf (step S408). Accordingly, the gripping force F decreases by ΔF, and the region R where the required gripping pressure is generated shifts further toward the distal end.

The processor 2b repeats the process from step S406 to step S408 until the closing driving force f decreases to a minimum closing driving force fmin.

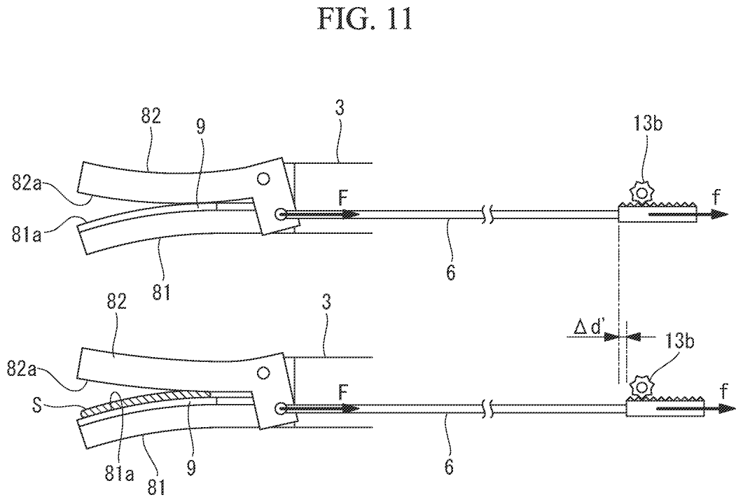

The predetermined threshold value Th and the decreasing amount Δf are set for each magnitude value of the closing driving force f. For example, as shown in FIG. 11, the threshold value Th is set based on a deviation amount Δd′ between the position of the proximal end of the driving-force transmission member 6 when the jaws 81 and 82 are closed by the closing driving force f and the tissue S is not gripped between the gripping surfaces 81a and 82a and the position of the proximal end of the driving-force transmission member 6 when the jaws 81 and 82 are closed by the closing driving force f and the tissue S is gripped between the gripping surfaces 81a and 82a. Thus, when the deviation amount Δd from the start of the incision process using the closing driving force f exceeds the predetermined threshold value Th, it can be determined that the incision process performed on the tissue S has progressed by a certain degree or more.

Alternatively, the predetermined threshold value Th and the decreasing amount Δf may be fixed regardless of the magnitude of the closing driving force f.

When the closing driving force f decreases to the minimum driving force fmin (YES in step S409) or when the operator operates the treatment operation section 14 to stop the incision process (NO in step S403), the processor 2b causes the energy unit 7 to stop supplying the energy source to the treatment section 9 and causes the driver 13 to stop temporally changing the closing driving force f (step S404). The minimum closing driving force fmin may be zero or a freely-chosen value larger than zero.

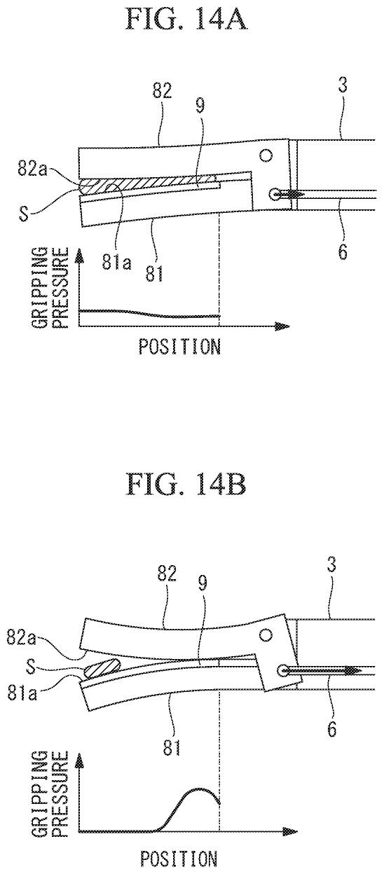

As shown in FIG. 14A, if the tissue S is thick, the gripping pressure is spatially distributed when the gripping force F is low, thus making it difficult to apply sufficient gripping pressure to the tissue S. Furthermore, as shown in FIG. 14B, if the tissue S is to be gripped by a part of the pair of gripping surfaces 81a and 82a, there may be timings when the gripping pressure is not applied to the tissue S.

In this embodiment, the treatment detector 15 is added to the configuration of the first embodiment, and the closing driving force f is gradually decreased in accordance with the detection result obtained by the treatment detector 15, so that the following advantages can be further achieved, as compared with the first embodiment.

Specifically, the incision process of the tissue S commences with the high gripping force Fmax, and the progress of the incision process performed on the tissue S is monitored based on the degree of opening between the pair of jaws 81 and 82 detected by the treatment detector 15. When it is detected that the incision process performed on the tissue S has progressed by a certain degree or more, the gripping force F decreases by ΔF, so that the region R where the required gripping pressure is generated shifts toward the distal end. Accordingly, by proceeding with the incision process of the tissue S in one direction from the proximal ends toward the distal ends of the gripping surfaces 81a and 82a while checking that the tissue S is incised, the tissue S can be incised more reliably and more efficiently over the entire length even if the tissue S is thick or the tissue S is gripped by a part of the pair of gripping surfaces 81a and 82a.

According to this embodiment, the tissue S can be incised within a shorter period of time, as compared with the first embodiment.

As an alternative to this embodiment in which the processor 2b gradually decreases the closing driving force f from the high driving force fmax, the processor 2b may gradually increase the closing driving force f to shift the region R in one direction from the distal ends toward the proximal ends of the pair of gripping surfaces 81a and 82a.

In a case where each jaws intersect each other like a pair of scissors, the relationship between the high driving force and the closing driving force is inverted. For example, when proceeding with the incision process of the tissue S from the proximal ends toward the distal ends of the gripping surfaces 81a and 82a, the closing driving force f is gradually increased from the low driving force fmin to the high driving force fmax.

### Third Embodiment

Next, a medical device and a control method therefor according to a third embodiment of the present invention will be described.

In this embodiment, components different from those in the first and second embodiments will be described, whereas components identical to those in the first and second embodiments will be given the same reference signs, and descriptions thereof will be omitted.

A medical device 300 according to this embodiment gradually decreases the closing driving force f from the high driving force fmax in accordance with the progress of the incision process, and causes the closing driving force f to temporally fluctuate after the positional deviation amount Δd of the proximal end of the driving-force transmission member 6 becomes smaller than or equal to a predetermined threshold value Th2. Specifically, the method for controlling the medical device according to this embodiment is a combination of the control method according to the first embodiment and the control method according to the second embodiment.

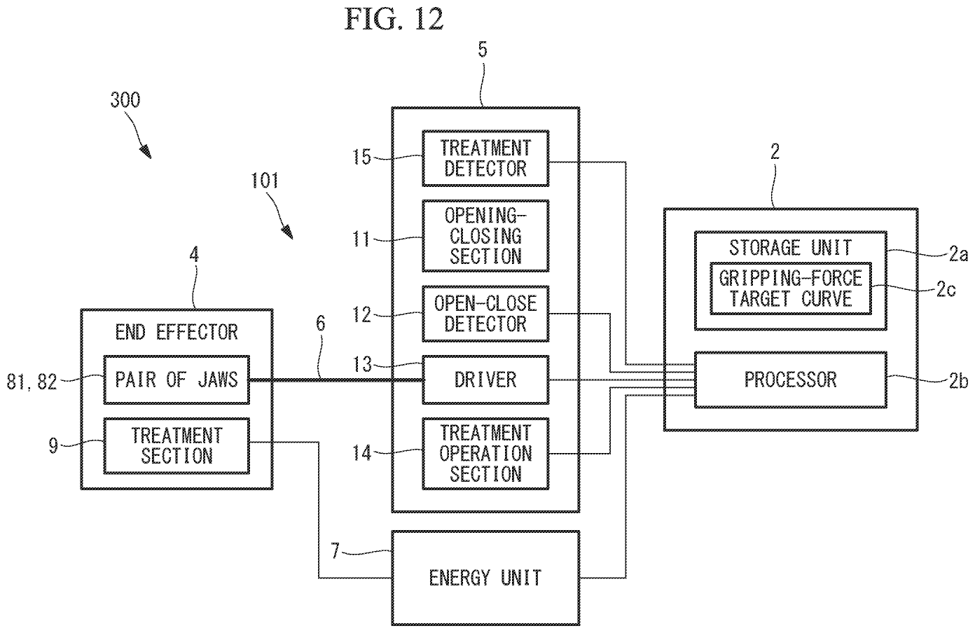

In detail, as shown in FIG. 12, the medical device 300 includes the device body 101 and the controller 2, and the gripping-force target curve 2c is stored in the storage unit 2a.

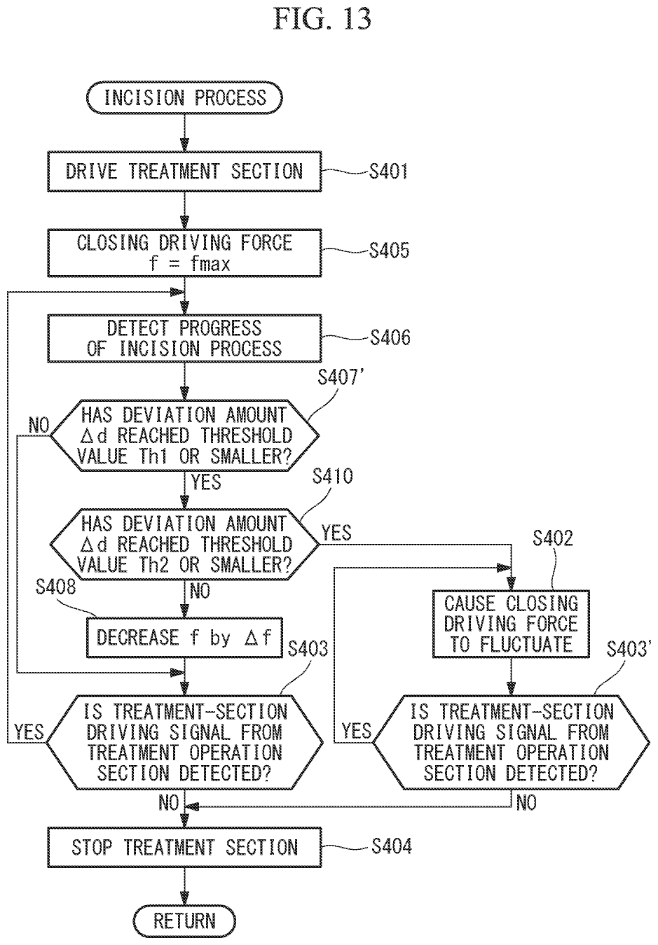

In this embodiment, as shown in FIG. 13, the processor 2b drives the treatment section 9 and sets the closing driving force f to the high driving force fmax in response to a treatment-section driving signal (step S401 and step S405). Then, the processor 2b monitors the position of the proximal end of the driving-force transmission member 6 detected by the treatment detector 15 during a period in which the tissue S is being incised by the treatment section 9 (step S406).

In this embodiment, the processor 2b calculates a positional deviation amount Δd of the proximal end of the driving-force transmission member 6 caused by the thickness of the tissue S between the gripping surfaces 81a and 82a. In other words, the deviation amount Δd is a positional deviation amount of the proximal end of the driving-force transmission member 6 detected by the encoder relative to the position of the proximal end of the driving-force transmission member 6 when the tissue S is not gripped between the pair of gripping surfaces 81a and 82a in a state where the pair of jaws 81 and 82 is closed by the closing driving force f. For example, the storage unit 2a has stored therein a correspondence relationship between the magnitude of the closing driving force f and the position of the proximal end of the driving-force transmission member 6 when the tissue S is not gripped between the pair of gripping surfaces 81a and 82a. Based on this correspondence relationship, the processor 2b calculates the deviation amount Δd from the current magnitude of the closing driving force f and the position of the proximal end of the driving-force transmission member 6 detected by the encoder.

The processor 2b compares the deviation amount Δd with a predetermined first threshold value Th1 to determine whether or not the incision process of the tissue S between the gripping surfaces 81a and 82a has progressed until the thickness of the tissue S has reached a predetermined thickness or smaller (step S407′).

If the deviation amount Δd is larger than the first threshold value Th1 (NO in step S407′), the processor 2b proceeds to step S403.

If the deviation amount Δd is smaller than or equal to the first threshold value Th1 (YES in step S407′), the processor 2b then compares the deviation amount Δd with a predetermined second threshold value Th2 (step S410). The second threshold value Th2 is smaller than the first threshold value Th1.

If the deviation amount Δd is larger than the second threshold value Th2 (NO in step S410), the processor 2b decreases the closing driving force f by Δf (step S408) and repeats step S406 and step S407′.

In contrast, if the deviation amount Δd is smaller than or equal to the second threshold value Th2 (YES in step S410), the processor 2b proceeds to step S402 and causes the magnitude of the closing driving force f to temporally fluctuate in accordance with the gripping-force target curve 2c (step S402). Accordingly, the remaining thin tissue S is incised over the entire length.

When the operator operates the treatment operation section 14 to terminate the incision process (YES in step S403 or step S403′), the processor 2b causes the energy unit 7 to stop supplying the energy source to the treatment section 9 and causes the driver 13 to stop temporally changing the closing driving force f (step S404).

For example, the second threshold value Th2 for the deviation amount Δd is set to a value twice as large as detection accuracy (resolving power) d0 of the encoder. The deviation amount Δd detected by the encoder and caused by the thickness of the tissue S is equal to (d1−d2)±2×d0, where d1 denotes the position of the proximal end of the driving-force transmission member 6 when the pair of jaws 81 and 82 is closed by the closing driving force f and the tissue S is not gripped between the pair of gripping surfaces 81a and 82a, and d2 denotes the position of the proximal end of the driving-force transmission member 6 when the pair of jaws 81 and 82 is closed by the closing driving force f and the tissue S is gripped between the pair of gripping surfaces 81a and 82a. Therefore, when the deviation amount Δd is smaller than or equal to 2×d0, it is difficult to accurately detect the progress of the incision process performed on the tissue S.

If the tissue S between the gripping surfaces 81a and 82a is thin, the positional deviation amount Δd of the proximal end of the driving-force transmission member 6 is small. It is difficult for the encoder to accurately detect a small deviation amount Δd. As a result, it is difficult to accurately detect the progress of the incision process performed on the thin tissue S, and also to detect whether or not the tissue S has been completely incised after the incision process has progressed by a certain degree or more.

In a case where a highly accurate encoder capable of detecting a small deviation amount Δd is employed, the cost of the device increases.

This embodiment is a combination of the second embodiment and the first embodiment, so that the following advantages can be further achieved, as compared with the second embodiment. Specifically, when the thickness of the tissue S between the gripping surfaces 81a and 82a is larger than a thickness equivalent to the detection accuracy of the encoder, the tissue S is incised in one direction as a result of the gripping force F gradually decreasing from the high gripping force Fmax. Then, after the thickness of the tissue S between the gripping surfaces 81a and 82a decreases to the thickness equivalent to the detection accuracy of the encoder, the gripping force F fluctuates between the high gripping force Fmax and the low gripping force Fmin, so that the thin tissue S remaining between the gripping surfaces 81a and 82a is incised over the entire length.

Accordingly, without having to use a special encoder with high detection accuracy, thin tissue S or thin tissue S remaining between the gripping surfaces 81a and 82a after the incision process has progressed by a certain degree or more can be incised more reliably over the entire length.

Moreover, in this embodiment, the tissue S can be incised within a shorter period of time, as compared with the first embodiment.

As an alternative to each of the above embodiments in which the driver 13 includes the motor 13a as a driving source that generates a driving force, another driving source, such as a piezoelectric element, may be provided.

As an alternative to each of the above embodiments in which the treatment detector 15 uses an encoder provided at the driver 13 for detecting the degree of opening between the pair of jaws 81 and 82, another device may be used. For example, the treatment detector 15 may use a visual sensor or an encoder that directly detects the swivel angle of the movable jaw 82. The visual sensor may be an endoscope used for observing the jaws 81 and 82. Alternatively, the treatment detector 15 may use the visual sensor, such as an endoscope, to directly detect the progress of the incision process performed on the tissue S between the gripping surfaces 81a and 82a.

As an alternative to the embodiments described above in which the medical devices 100, 200, and 300 are of a type that is directly gripped by the hand of the operator, the medical device according to the present embodiment may also be applied to a medical master-slave system. Specifically, the device body 1 serving as a slave device may be connected to a master device having the processor 2b, and the treatment section 9 and the driver 13 of the device body 1 may operate in accordance with a control signal from the master device. In this case, the master device in place of the device body 1 may be provided with the opening-closing section 11, the open-close detector 12, and the treatment operation section 14.

The following aspects can be also derived from the embodiments.

An aspect of the present embodiment provides a medical device including: a pair of jaws that grips biological tissue, is coupled to each other at first ends of the pair of jaws, opens and closes as a result of swiveling of at least one of the jaws, and comprises a pair of gripping surfaces facing each other when the pair of jaws is closed; a driving source that generates a closing driving force for closing the pair of jaws; a driving-force transmission member that connects the pair of jaws to the driving source and that transmits the closing driving force from the driving source to at least one of the pair of jaws; a treatment section that is provided on at least one of the pair of jaws and that treats the biological tissue; and a control unit that is connected to the driving source and that controls the driving source. A region where gripping pressure is generated between the pair of gripping surfaces shifts between first ends and second ends of the pair of gripping surfaces in accordance with magnitude of the closing driving force. The control unit temporally changes the magnitude of the closing driving force generated by the driving source so as to shift the region where the gripping pressure is generated.

According to this aspect, the closing driving force generated by the driving source is transmitted to the pair of jaws via the driving-force transmission member, so that the pair of jaws close, whereby the biological tissue is gripped between the gripping surfaces of the pair of jaws. In this state, the biological tissue can be treated by the treatment section.

In order to treat the biological tissue, the biological tissue can be gripped by the pair of gripping surfaces with a certain gripping pressure or more. The bending of the jaws increases with increasing closing driving force, such that the region where the gripping pressure is generated shifts from the first end toward the second end. Thus, the position where the biological tissue is efficiently treated varies depending on the magnitude of the closing driving force.

According to this aspect, the control unit temporally changes the closing driving force generated by the driving source in a state where the pair of jaws are closed. Accordingly, a certain gripping pressure or more can be generated over the entire length of the gripping surfaces, so that the biological tissue between the gripping surfaces can be treated over the entire length. Moreover, since the region where the gripping pressure is generated is shifted by utilizing the bending of the jaws, jaws having a small diameter can be favorably utilized. Accordingly, it is possible to reduce the diameter of the jaws while also generating gripping pressure over the entire length of the gripping surfaces.

In the above aspect, the treatment section may apply energy for incising the biological tissue to the biological tissue between the pair of gripping surfaces.

According to this configuration, the energy is applied to the biological tissue from the treatment section while the closing driving force is temporally changed, so that the biological tissue between the pair of gripping surfaces can receive the energy and be treated over the entire length.

In the above aspect, the control unit may temporally change the magnitude of the closing driving force during a period in which the treatment section treats the biological tissue.

According to this configuration, the driving source is automatically controlled in synchronization with the driving of the treatment section, so that the region where the gripping pressure is generated can be automatically shifted simultaneously with the treatment performed on the biological tissue by the treatment section.

In the above aspect, the control unit may temporally change the magnitude of the closing driving force in accordance with a predetermined temporal change pattern.

According to this configuration, desired treatment can be performed on the biological tissue in accordance with a simple device and simple control.

In the above aspect, the magnitude of the closing driving force in the predetermined temporal change pattern may fluctuate between a first driving force in which the gripping pressure is generated at the first ends of the pair of gripping surfaces and a second driving force in which the gripping pressure is generated at the second ends of the pair of gripping surfaces.

According to this configuration, the biological tissue between the pair of gripping surfaces can be treated more reliably over the entire length.

In the above aspect, the medical device may further include a storage unit that stores a gripping-force target curve indicating a temporal change pattern of a gripping force applied to the pair of jaws from the driving-force transmission member. The gripping force is based on the closing driving force. The control unit may temporally change the magnitude of the closing driving force in accordance with the gripping-force target curve stored in the storage unit.

According to this configuration, the closing driving force is temporally changed in accordance with the gripping-force target curve, so that a gripping-pressure spatial distribution designed in advance can be generated between the gripping surfaces, whereby desired treatment can be performed on the biological tissue more reliably.

In the above aspect, the medical device may further include a treatment detector that detects a degree of opening between the pair of jaws.

The degree of opening between the pair of jaws indicates the thickness of the biological tissue between the pair of gripping surfaces. If the thickness of the biological tissue between the pair of gripping surfaces changes as the treatment performed on the biological tissue by the treatment section progresses, the progress of the treatment performed on the biological tissue can be estimated based on the degree of opening detected by the treatment detector.

In the above aspect, the control unit may gradually decrease or gradually increase the closing driving force in accordance with the degree of opening detected by the treatment detector.

According to this configuration, as the treatment performed on the tissue progresses, the region where the gripping pressure is generated shifts in one direction from the first ends toward the second ends of the pair of gripping surfaces or from the second ends toward the first ends. Accordingly, the biological tissue between the pair of gripping surfaces can be treated more reliably and more efficiently.

In the above aspect, after the degree of opening between the pair of jaws detected by the treatment detector becomes smaller than or equal to a predetermined threshold value, the control unit may cause the magnitude of the closing driving force to fluctuate between a first driving force in which the gripping pressure is generated at the first ends of the pair of gripping surfaces and a second driving force in which the gripping pressure is generated at the second ends of the pair of gripping surfaces.

The degree of opening between the pair of jaws decreases with decreasing thickness of the biological tissue between the pair of gripping surfaces, resulting in a decrease in the detection accuracy with respect to the degree of opening. Therefore, after the biological tissue decreases in thickness with the progression of the treatment, the region where the gripping pressure is generated is caused to reciprocate between the first ends and the second ends of the pair of gripping surfaces, so that the thin biological tissue remaining between the pair of gripping surfaces can be treated more reliably over the entire length.

Another aspect of the present embodiment provides a method for controlling a medical device including a pair of jaws that grips biological tissue. The pair of jaws is coupled to each other at first ends of the pair of jaws and opens and closes as a result of swiveling. The method includes a step for temporally changing the magnitude of a closing driving force for closing the pair of jaws.

In the above aspect, the method may further include a step for applying energy for incising the biological tissue to the biological tissue between the pair of jaws.

In the above aspect, the step for temporally changing the magnitude of the closing driving force may be performed during a period in which a step for treating the biological tissue is being performed.

In the above aspect, the step for temporally changing the magnitude of the closing driving force may include a step for temporally changing the magnitude of the closing driving force in accordance with a predetermined temporal change pattern. The magnitude of the closing driving force in the predetermined temporal change pattern may fluctuate between a first driving force in which gripping pressure is generated at first ends of a pair of gripping surfaces and a second driving force in which the gripping pressure is generated at second ends of the pair of gripping surfaces.

In the above aspect, the step for temporally changing the magnitude of the closing driving force in accordance with the predetermined temporal change pattern may include a step for temporally changing the magnitude of the closing driving force in accordance with a gripping-force target curve stored in a storage unit. The gripping-force target curve may indicate a temporal change pattern of a gripping force that is applied to the pair of jaws and that is based on the closing driving force.

In the above aspect, the method may further include a step for detecting a degree of opening between the pair of jaws. In this configuration, the step for temporally changing the magnitude of the closing driving force may include a step for gradually decreasing or gradually increasing the closing driving force in accordance with the detected degree of opening. Furthermore, in this configuration, after the detected degree of opening becomes smaller than or equal to a predetermined threshold value, the magnitude of the closing driving force may fluctuate between a first driving force in which gripping pressure is generated at first ends of a pair of gripping surfaces and a second driving force in which the gripping pressure is generated at second ends of the pair of gripping surfaces.

### REFERENCE SIGNS LIST

2a storage unit
2b processor (control unit)
2c gripping-force target curve
6 driving-force transmission member
81, 82 jaw
81a, 82a gripping surface
9 treatment section
13a motor (driving source)
15 treatment detector
100, 200, 300 medical device
S biological tissue

1. A medical device comprising:
a pair of jaws that grips biological tissue, is coupled at first ends of the pair of jaws, opens and closes as a result of swiveling of at least one of the jaws, and comprises a pair of gripping surfaces facing when the pair of jaws is closed;
a motor that generates a closing driving force for closing the pair of jaws;
a driving-force transmission member that connects the pair of jaws to the motor and that is configured to transmit the closing driving force from the motor to at least one of the pair of jaws; and
a controller that is connected to the motor and that controls the motor,
wherein the controller temporally changes magnitude of the closing driving force generated by the motor so as to shift peak positions of gripping pressure for gripping the biological tissue in the pair of gripping surfaces.

2. The medical device according to claim 1, wherein
the controller causes the magnitude of the closing driving force to be transited between a first driving force for which first ends of the pair of gripping surfaces becomes the peak positions and a second driving force for which second ends, which are opposite to the first ends, of the pair of gripping surfaces becomes the peak positions.

3. The medical device according to claim 2, wherein
the controller fluctuates the magnitude of the closing driving force so that one of the first driving force and the second driving force is served as a maximum value and so that the other of the first driving force and the second driving force is served as a minimum value.

4. The medical device according to claim 2, further comprising a treatment section, wherein
the treatment section is provided on at least one of the pair of gripping surfaces and outputs energy for incising the biological tissue, and
the controller temporally changes the magnitude of the closing driving force during a period in which the treatment section outputs the energy.

5. The medical device according to claim 4, wherein
the controller stores a target value indicating a temporal change pattern of a gripping force applied to the pair of jaws from the driving-force transmission member, the gripping force being based on the closing driving force, and
the controller temporally changes the magnitude of the closing driving force in accordance with the stored target value.

6. The medical device according to claim 5, further comprising a treatment detector that detects a degree of opening between the pair of jaws, wherein
the controller gradually decreases or gradually increases the closing driving force in accordance with the degree of opening detected by the treatment detector.

7. The medical device according to claim 6, wherein
after the degree of opening detected by the treatment detector becomes smaller than or equal to a predetermined threshold value, the controller causes the magnitude of the closing driving force to be transited between the first driving force and the second driving force.

8. A method for controlling a medical device including a pair of jaws that grips biological tissue, comprises a pair of gripping surfaces facing when the pair of jaws is closed, the method comprising:
temporally changing magnitude of a closing driving force for closing the pair of jaws so as to shift peak positions of gripping pressure for gripping the biological tissue in the pair of gripping surfaces.

9. The method for controlling the medical device according to claim 8, wherein
in the temporally changing magnitude of the closing driving force, causing the magnitude of the closing driving force to be transited between a first driving force for which first ends of the pair of gripping surfaces becomes the peak positions and a second driving force for which second ends, which are opposite to the first ends, of the pair of gripping surfaces becomes the peak positions.

10. The method for controlling the medical device according to claim 9, wherein
in the temporally changing magnitude of the closing driving force, fluctuating the magnitude of the closing driving force so that one of the first driving force and the second driving force is served as a maximum value and so that the other of the first driving force and the second driving force is served as a minimum value.

11. The method for controlling the medical device according to claim 9, wherein
the medical device includes a treatment section, and
the temporally changing magnitude of the closing driving force is performed during a period in which the treatment section output a energy.

12. The method for controlling the medical device according to claim 11, further comprising temporally changing the magnitude of the closing driving force in accordance with a target value stored in a storage unit.

13. The method for controlling the medical device according to claim 12, further comprising:
detecting a degree of opening between the pair of jaws; and
gradually decreasing or gradually increasing the closing driving force in accordance with the detected degree of opening.

14. The method for controlling the medical device according to claim 13, further comprising:
after the detected degree of opening becomes smaller than or equal to a predetermined threshold value, causing the magnitude of the closing driving force to be transited between the first driving force and the second driving force.

15. A control device controlling a medical system that includes a pair of jaws that grips biological tissue, is coupled at first ends of the pair of jaws, opens and closes as a result of swiveling of at least one of the jaws, and comprises a pair of gripping surfaces facing when the pair of jaws is closed, a motor that generates a closing driving force for closing the pair of jaws and a driving-force transmission member that connects the pair of jaws to the motor and that is configured to transmit the closing driving force from the motor to at least one of the pair of jaws, the control device comprising:
a processor comprising hardware, wherein
the processor is configured to temporally change magnitude of the closing driving force generated by the motor so as to shift peak positions of gripping pressure for gripping the biological tissue in the pair of gripping surfaces.

16. The control device according to claim 15, wherein
the processor is configured to cause the magnitude of the closing driving force to be transited between a first driving force for which first ends of the pair of gripping surfaces becomes the peak positions and a second driving force for which second ends, which are opposite to the first ends, of the pair of gripping surfaces becomes the peak positions.

17. The control device according to claim 16, wherein
the processor is configured to fluctuate the magnitude of the closing driving force so that one of the first driving force and the second driving force is served as a maximum value and so that the other of the first driving force and the second driving force is served as a minimum value.

18. The control device according to claim 17, wherein
the medical system includes a treatment section,
the treatment section is provided on at least one of the pair of gripping surfaces and outputs energy for incising the biological tissue, and
the processor is configured to temporally change the magnitude of the closing driving force during a period in which the treatment section outputs the energy.

19. The control device according to claim 18, further comprising a memory that stores a target value indicating a temporal change pattern of a gripping force applied to the pair of jaws from the driving-force transmission member, the gripping force being based on the closing driving force, wherein
the processor is configured to temporally change the magnitude of the closing driving force in accordance with the target value stored in the memory.

20. The control device according to claim 19, further comprising a treatment detector that detects a degree of opening between the pair of jaws, wherein
the processor is configured to gradually decrease or gradually increase the closing driving force in accordance with the degree of opening detected by the treatment detector.
